(19) United States
(12) Patent Application Publication
(10) Pub. No.: US 2010/0112022 A1
(43) Pub. Date: May 6, 2010

(54) Antiperspirant Products and Methods of Merchandising the Same

(76) Inventors: Jody Lynn Hoying, Maineville, OH (US); David Frederick Swaile, Cincinnati, OH (US); Athena Kasvikis, Cincinnati, OH (US); Julane Kirtley Becker, Ludlow, KY (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/562,014

(22) Filed: Sep. 17, 2009

Related U.S. Application Data
(60) Provisional application No. 61/097,843, filed on Sep. 17, 2008.

(51) Int. Cl. A61K 8/97; A61K 8/02; A61Q 15/00; G06Q 90/00

(52) U.S. Cl. 424/401; 424/65; 705/500

(57) ABSTRACT

A method of merchandising an antiperspirant product, comprising the steps of offering a solid or cream antiperspirant product for sale, wherein the antiperspirant product is a water-in-oil emulsion comprising 5% or more of water and one or more plant-derived materials in the continuous oil phase and communicating to perspective buyers that the antiperspirant product is natural and/or provides an environmental benefit.

### CROSS REFERENCE TO RELATED APPLICATION

This application claims the benefit of U.S. Provisional Application No. 61/097,843, filed Sep. 17, 2008.

### FIELD OF THE INVENTION

The present invention is directed to antiperspirant products, methods of merchandising antiperspirant products, and underarm product line-ups including both antiperspirant products and deodorant products.

### BACKGROUND OF THE INVENTION

Consumers are becoming more conscientious about purchasing and using “natural” and “environmentally friendly” products. Consumers however are not willing to sacrifice performance for a more natural alternative, particularly when it comes to their personal care products. For example, there is a host of so-called natural antiperspirant and deodorant products being marketed, but these products often provide inferior wetness protection compared to traditional antiperspirant products that contain a metal salt antiperspirant active.

Developers and marketers of “natural” or “environmentally friendly” products generally tend to make products that focus only on the non-use attributes, without significantly taking in-use performance into account. The development approach typically addresses how the product benefits nature and the environment, with little concern whether or not the product actually works and provides the in-use benefits for which it is marketed. Thus, the market place often provides alternative products at extreme ends of a continuum spanning traditional antiperspirant products and natural products for underarm care. What is needed however is a balance; an antiperspirant product that employs some “natural ingredients” in place of traditional ingredients and that provides comparable in-use performance. And the natural, environmental, or other differing characteristics associated with such an antiperspirant product needs to be communicated to perspective buyers so that they can make an informed purchase decision when faced with the plethora of antiperspirant options available on a typical store shelf.

### DETAILED DESCRIPTION OF ILLUSTRATIVE EMBODIMENTS OF THE INVENTION

The present invention may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

All percentages and ratios used herein are by weight of the total composition, and all measurements made are at 25° C., unless otherwise designated.

The compositions/methods of the present invention can comprise, consist of, and consist essentially of the features and/or steps of the invention described herein, as well as any of the additional or optional ingredients, components, steps, or limitations described herein.

The term “ambient conditions” as used herein refers to surrounding conditions at about one atmosphere of pressure, about 50% relative humidity and about 25° C.

The term “water-immiscible” as used herein refers to materials or mixtures of materials with less than 1% water solubility at 25° C., and preferably less than 0.1% water solubility at 25° C. Most preferable are materials with less than 0.01% water solubility at 25° C.

The term “volatile” as used herein refers to those materials which have a measurable vapor pressure as measured at 25° C. and 1 atmosphere. The term “moderately volatile material,” as used herein, refers to those materials with a vapor pressure below about 2 mmHg at 25° C. The term “low volatile material,” as used herein, refers to those materials with a vapor pressure below about 0.5 mmHg at 25° C. The term “nonvolatile material,” as used herein, refers to those materials with a vapor pressure below about 0.002 mmHg at 25° C. Vapor pressures can be measured in a variety of manners and are often available in a variety of chemical data bases that would be known to one skilled in the art. One such database is available from the Research Institute for Fragrance Materials.

The term “plant-derived” material as used herein means a material that was originally harvested as a plant and then subsequently modified, chemically and/or physically.

The phrase “labeled CEPA toxic” as used herein refers to materials on the Canadian Environmental Protection Act's list of toxic materials and those materials on the CEPA's Priority Substances List that are ultimately found to be toxic after their assessment.

The terms “light” and “weightlessness” as used herein include these words themselves and all synonyms thereof.

### I. Methods of Merchandising

The present invention is directed to methods of merchandising antiperspirant products that have a specified characteristic and/or provide a specified benefit which is communicated to perspective buyers in conjunction with offering the antiperspirant product for sale. The communication step of the merchandising methods of the present invention may be accomplished in a variety of ways. The communication may be done, for example, via a printed substrate. The printed substrate can be employed as part or all of the primary and/or secondary packaging of the antiperspirant product. “Primary packaging,” as used herein, means any container in which the beauty care product is in direct contact. And “secondary packaging” means any additional materials that are associated with the primary packaging, such as, for example, a box or polymeric sleeve that at least partially surrounds the primary packaging. The printed substrate may also be a brochure, instruction sheet, or other informative document that is associated with the antiperspirant product. Although not required by the present invention, these documents are typically included in or attached (permanently or temporarily) to packaging materials.

Advertisement is another exemplary method of communicating product information to perspective buyers. Illustrative advertisement media includes television/internet broadcasts, a page in a magazine or newspaper, a sticker, a coupon, a flyer, an in-aisle or end-of-aisle display, and point-of-sale items intended to either be taken by prospective buyers or remain in an area proximate the antiperspirant product. It should be noted that if advertisement which is disconnected from the antiperspirant product itself is utilized, then the communication provided in the advertisement may or may not be reproduced on packaging of the antiperspirant product.

The antiperspirant product characteristic and/or benefit that are communicated through the methods provided herein can also vary. One exemplary communication is that the antiperspirant product is natural and/or it provides an environmental benefit. In this context, the communication can include terms such as the following: natural, nature, plant, vegetable, environment, eco, green, pure, purity, not artificial, organic, not man-made, petroleum free, being, wellness, health, and synonyms thereof. Another exemplary communication is that the antiperspirant product provides a feel and appearance aesthetic benefit by communicating terms such as, for example, light, weightlessness, clear, clean, and synonyms thereof.

The communication aspects discussed above are functionally linked to the associated antiperspirant product due to the choice of ingredients employed and/or concentration level of ingredients, as compared to existing marketed antiperspirant products. For methods of merchandising that include a communication that the associated antiperspirant product is natural and/or it provides an environmental benefit, the following non-limiting list of formulation parameters can be chosen as the basis for the communication: 1) inclusion and level of water (e.g., 5%, 10%, 15%, 20%, 25%, 30%, 35%, 40% or more); 2) level of aluminum contained in the antiperspirant active (e.g., 1%, 2%, 2.5%, 3%, 4%, 1-4%, and 1-3%); 3) the inclusion of plant-derived materials (e.g., rheology-modifying material, oil, emollient, and colorant); 4) inclusion and level of volatile silicone that is not labeled as CEPA toxic; and 5) combinations thereof.

For methods of merchandising that include a communication that the associated antiperspirant product provides a “light” or “weightlessness” benefit, the level of water and/or level of volatile materials employed can be chosen so that the volume of antiperspirant composition dispensed on one's underarm does not feel heavy or cakey. For example, the associated antiperspirant product can contain at least about 5%, 10%, 15%, 20%, 25%, 30%, 35%, or 40% water. The concentration of volatile materials may be at least about 50%, 55%, 60%, 65%, 70%, or 75%, for example. The inclusion of plant-derived or natural materials may also provide a light or weightlessness perception.

Antiperspirant products can be formulated into a number of different final product forms, including, for example, liquids, clear gels, solid sticks, and creams (can be commonly referred to as “soft solids”). The antiperspirant products of the present invention are preferably a water-in-oil emulsion composition, either in a cream form or a solid form. The emulsion antiperspirant product comprises a continuous phase and a disperse aqueous phase. The continuous phase includes one or more water-immiscible liquids and an optional rheology-modifying material. The disperse phase includes an antiperspirant active dissolved in a polar solvent. These and optional components will be described in more detail below.

### II. Water-in-oil Emulsion Antiperspirant Compositions

A. Continuous Phase

1. Water-Immiscible Liquid

The concentration of the water-immiscible liquid preferably ranges from about 10% to about 30%, by weight of the composition. Other concentrations however are also contemplated herein.

One preferred water-immiscible liquid that may be employed in exemplary antiperspirant compositions that can be made in accordance with the present invention comprises volatile silicones, non-volatile silicones, or mixtures of these materials. Nonlimiting examples include those volatile silicones that are described in Todd et al., “Volatile Silicone Fluids for Cosmetics”, Cosmetics and Toiletries, 91:27-32 (1976). Suitable amongst these volatile silicones include the cyclic silicones having from about 3 or from about 4 to about 7 or to about 6, silicon atoms. Specifically are those which conform to the formula:

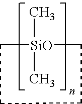

wherein n is from about 3, from about 4 or about 5 to about 7 or to about 6. These volatile cyclic silicones generally have a viscosity value of less than about 10 centistokes. Other suitable water-immiscible liquids for use herein include those volatile and nonvolatile linear silicones which conform to the formula:

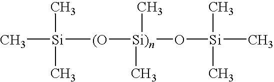

wherein n is greater than or equal to 0. The volatile linear silicone materials will generally have viscosity values of less than 5 centistokes at 25° C. The non-volatile linear silicone materials will generally have viscosity values of greater than 5 centistokes at 25° C.

Specific examples of suitable volatile silicones for use herein include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™(available from Mazer); and mixtures thereof. Examples of preferred volatile silicones include cyclohexamethylsiloxane, hexyl methicone, capryl methicone and linear or branched polydimethyl siloxanes containing 4 to 6 silicone atoms.

As noted above, one of the formulation levers that can be used to create a more natural or environmentally focused antiperspirant product is the use of volatile silicones that are not labeled CEPA toxic. Furthermore, the concentration level of such a material can be adjusted. For example, antiperspirant products of the present invention can employ less than about 30% of a volatile silicone that is not labeled CEPA toxic, including 5%, 10%, 15%, 20%, 25% or ranges incorporating one of these values.

Specific examples of suitable non-volatile linear silicones for use herein include, but are not limited to, Rhodorsil Oils 70047 available from Rhone-Poulenc; Masil SF Fluid available from Mazer; Dow Corning 200 and Dow Corning 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones).

Other suitable non-volatile silicone materials that may be employed in antiperspirant compositions manufacturable by the present invention include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. These non-volatile silicone liquid carriers will generally have viscosity values of less than about 100,000 centistokes, less than about 500 centistokes, or from about 1 centistokes to about 200 centistokes or to about 50 centistokes, as measured under ambient conditions.

Silicon-free hydrophobic liquids can be employed alternatively or additionally to liquid silicones. Examples of silicon-free hydrophobic liquids include aliphatic hydrocarbons such as mineral oils, hydrogenated polyisobutane, polydecene, paraffins, isoparaffins, and aliphatic ethers derived from at least one fatty alcohol (e.g., PPG-3 myristeyl ether and PPG-14 butyl ether).

Other hydrophobic liquids include aliphatic or aromatic esters. Exemplary aliphatic esters contain at least one long chain alkyl group, such as ester derived from C1 to C20 alkanols esterified with a C8 to C22 alkanoic acid or C6 to C10 alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate. Exemplary aromatic esters include fatty alkyl benzoates.

Plant-derived oils can be used in the in the continuous phase as either the predominant water-immiscible carrier liquid or as a minor component thereof. Exemplary plant-derived oils include almond oil, avocado oil, coconut oil, hazelnut oil, jojoba oil, olive oil, palm oil, pumpkin seed oil, safflower oil, sesame oil, sunflower oil, tamanu oil, wheat germ oil, castor oil, cottonseed oil, and grapeseed oil. In one embodiment, plant-derived oils can be employed at levels above 2%, 5%, or even 10%.

Water-immiscible liquids other than those disclosed above may also be employed by the present invention. Further, it is to be understood that the continuous phase may contain hydrophilic materials, so long as the continuous phase overall is water-immiscible.

2. Rheology Modifying Materials

Rheology modifying materials can be employed to drive the desired product viscosity or hardness. The rheology modifying materials can comprise structurants, gellants, thickeners, and the like. Suitable rheology modifying materials include polyethylene waxes, ozokerite waxes, candellia waxes, carnauba waxes, and mixtures thereof. Other suitable structurant materials include N-acyl amino acid amides and esters; for example, N-Lauroyl-L-glutamic acid di-n-butylamide. These materials are described in greater detail in U.S. Pat. No. 3,969,087. 12-hydroxystearic acid and esters and amines of the same represent another class of useful rheology modifying materials for the antiperspirant compositions of the present invention.

Fiber-forming structurants may also be employed. These materials create a network of fibers or strands that extend throughout the continuous phase to gel the liquids therein. Such materials are generally non-polymeric, being monomers or dimmers that can have a molecular weight below about 10,000. Exemplary fiber-forming structurant materials have been reviewed by Terech and Weiss in “Low Molecular Mass Gelators of Organic Liquids and the Properties of their Gels” Chem. Rev 97, 3133-3159 [1997] and by Terech in Chapter 8, “Low-molecular Weight Organogelators” of the book “Specialist Surfactants” edited by I. D. Robb, Blackie Academic Professional, 1997.

Another suitable rheology modifying materials is a partially or fully esterified cellobiose according the following formula:

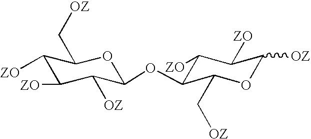

wherein each Z is independently hydrogen or an acyl group of the formula:

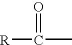

where R denotes a hydrocarbyl group containing from 4 to 22 carbon atoms. It one embodiment, not more than half of the Z groups are hydrogen.

Other suitable rheology modifying materials for use in the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, and triglycerides.

Suitable thickening or structuring agents can include, but are not limited to, solid salts of fatty acids wherein the fatty acid moiety has from about 12, from about 16 or from about 18 carbon atoms to about 40, to about 22, or about 20 carbon atoms. Suitable salt forming cations for use with these thickening or structuring agents include metal salts such as alkali metals (e.g. sodium and potassium), alkaline earth metals (e.g. magnesium), and aluminum. Preferred are sodium, potassium and aluminum salts. For example, suitable salt forming cations may be selected from the group consisting of sodium stearate, sodium palmitate, potassium stearate, potassium palmitate, sodium myristate, aluminum monostearate, and combinations thereof.

In some embodiments, natural or plant-derived rheology materials are employed as the predominant rheology modifying material. A representative, non-limiting list of such materials includes beeswax, cocoa butter, shea butter, carnauba wax, candellia wax, locust bean gum, guar gum, acacia gum, and clay minerals.

B. Disperse Phase

The disperse phase generally includes water and an antiperspirant active dissolved in a polar solvent, such as, for example, water ethanol or a liquid polyol. The concentration of the antiperspirant active in the composition should be sufficient to provide the finished antiperspirant product with the desired perspiration wetness and odor control. Exemplary antiperspirant active concentrations range include from about 0.1% to about 26%, from about 1% to about 20%, and from about 2% to about 10%, by weight of the composition. All such weight percentages are calculated on an anhydrous metal salt basis exclusive of water and any complexing or buffering agent such as, for example, glycine, glycine salts.

Suitable antiperspirant actives for use in the antiperspirant compositions of the present invention may include any compound, composition or other material having antiperspirant activity. Antiperspirant actives may include astringent metallic salts, especially the inorganic and organic salts of aluminum, zirconium and zinc, as well as mixtures thereof. Particularly beneficial are believed to be salts such as aluminum halides, aluminum chlorohydrate, aluminum hydroxyhalides, zirconyl oxyhalides, zirconyl hydroxyhalides, and mixtures thereof.

Aluminum salts for use in the antiperspirant compositions may include those that conform to the formula:

Al₂(OH)ₐCl_{b}.xH₂O

wherein a is from about 2 to about 5; the sum of a and b is about 6; x is from about 1 to about 6; and wherein a, b, and x may have non-integer values. One example is the aluminum chlorohydrates referred to as “⅚ basic chlorohydrate”, wherein a=5, and “⅔ basic chlorohydrate” wherein a=4. Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; and U.S. Pat. No. 4,359,456, Gosling et al., issued Nov. 16, 1982. Mixtures of aluminum salts are described in British Patent Specification 1,347,950, Shen et al., published Feb. 27, 1974.

Zirconium salts for use in the antiperspirant compositions may include those that conform to the formula:

ZrO(OH)₂₋ₐClₐ.xH₂O

wherein a is any number having a value of from 0 to about 2; x is from about 1 to about 7; and wherein a and x may both have non-integer values. Zirconium salts that additionally contain aluminum and glycine, commonly known as ZAG complexes, may also be used. These ZAG complexes contain aluminum chlorhydroxide and zirconyl hydroxy chloride conforming to the above-described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; U.S. Pat. No. 4,120,948, Shelton, issued Oct. 17, 1978 and U.S. Pat. No. 6,136,302, Juneja, issued Oct. 24, 2000.

Specific antiperspirant actives may include aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum sulfate buffered, aluminum zirconium trichlorohydrate, aluminum zirconium tretrachlorohydrate, aluminum zirconium pentachlorohydrate, aluminum zirconium octachlorohydrate, aluminum zirconium trichlorohydrdrex glycine, aluminum zirconium tretrachlorohydrex glycine, aluminum zirconium pentachlorohydrex glycine, aluminum zirconium octachlorohydrex glycine and combinations thereof.

One preferred species of antiperspirant actives includes aluminum only and aluminum-zirconium actives having a combined peak 4 and peak 5 area percentage of at least about 25% relative to the area sum of peaks 1 to 5, as determined by the gel permeation chromatography (“GPC”) methodology described below. Such preferred actives are disclosed in U.S. Pat. Nos. 6,245,325; 6,902,723; and 6,923,952. The antiperspirant actives employed in antiperspirant compositions herein can contain a stabilizer such as, for example, a calcium salt, a strontium salt, or mixture thereof, to maintain their efficacy during and after their manufacture. The GPC methodology can be performed as follows: the antiperspirant active salt samples are dissolved in 0.01 M nitric acid (which is also used as the mobile phase for the analysis) and chromatographed using 5 μl injections in a series of three consecutive Waters μ Porasil Columns, 3.9×300 mm, 10 μm packing. Samples should be diluted to produce an approximately 1% solution of active. A 1 mL per minute flow rate is recommended. Chromatograms are visualized using a Waters 410 Differential Refractometer. Samples are prepared immediately prior to analysis to prevent degradation. Relative peak areas and area ratios are calculated using a Waters Millennium Data System (Version 2.10 or equivalent). The peaks observed in the chromatogram are designated in order of appearance on the chromatogram as peaks 1-2 (appears as a single peak) and peaks 3, 4 and 5. The area of peaks 3, 4 and 5 correspond to the relative concentration of aluminum polymer species exiting the column during the specified time period from the injected sample. For aluminum and zirconium actives the area of peaks 1-2 corresponds to the relative concentration of co-eluting aluminum and zirconium polymer species appearing initially on the chromatogram.

Prior to any analysis, the columns should be conditioned individually by repeated 100 μl injections of a 10% zirconium-aluminum trichlorohydrate glycine solution (containing at least 10% zirconium on a solid basis). Conditioning is complete when the area percent of peaks 1-2 become relatively constant. During the conditioning process, the area percent of peaks 1-2 will increase, and there will be reduction in retention for all peaks. Columns should be discarded when peaks 1 and 2 are no longer resolved from peak 3.

The salts for the present invention may exhibit a combined peak 4 and/or peak 5 level that is greater than 25% of the total area of the chromatogram and preferably more than 30%. It should be noted, and known to one skilled in the art, that for aluminum only actives (i.e. aluminum chlorohydrate) may not contain any peak 1-2 so peak identification should be made by comparison to an appropriate standard.

In some embodiments, the level of aluminum contained in the antiperspirant product is reduced as compared to traditional antiperspirant products. And the antiperspirant product is communicated as being natural and/or providing an environmental benefit. In these embodiments, the level of aluminum is from about 1% to about 4% or from about 1% to about 3% (e.g, 2.5%).

The antiperspirant compositions provided herein may additionally employ a deodorant active. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

The disperse phase may optionally contain other polar materials. A representative, non-limiting list of optional polar materials includes C1 to C20 monohydric alcohols; C2 to C40 dihydric or polyhydric alcohols; alkyl ethers of all such alcohols, e.g., C1-C4 alkyl ethers; polyalkoxylated glycols, e.g., propylene glycols and polyethylene glycols having from 2 to 30 repeating alkoxylate (e.g., ethoxylate or propoxylate) groups and polyglycerols having from 2 to 16 repeating glycerol moieties; and mixtures thereof. More particular exemplary polar materials include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, 1,2 hexanediol and combinations thereof. It is to be understood that polar materials other than those listed above may also be employed in the antiperspirant compositions described herein.

C. Surfactants

Emulsifying surfactants are employed in the antiperspirant compositions to facilitate the formation of a stable emulsion containing the above-described continuous phase and disperse phase. The emulsifying surfactants may be anionic, cationic, zwitterionic and/or nonionic surfactants. Nonionic surfactants are preferred in the current invention. The proportion of emulsifier in the composition is often selected in the range up to 10% by weight and in many instances from 0.1 or 0.25 up to 5% by weight of the composition. Most preferred is an amount from 0.1 or 0.25 up to 3% by weight. Emulsifiers are frequently classified by HLB value. It is desirable, although not required, to use an emulsifier or a mixture of emulsifiers with an overall HLB value in a range from 2 to 10 preferably from 3 to 8.

It may be convenient to use a combination of two or more emulsifiers which have different HLB values above and below the desired value. By employing the two emulsifiers together in appropriate ratio, it is readily feasible to attain a weighted average HLB value that promotes the formation of an emulsion.

Many suitable emulsifiers of high HLB are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil, sunflower seed oil or soya bean oil. Such nonionic surfactants can also be derived from a polyhydroxy compound such as glycerol or sorbitol or other alditols. Examples of emulsifiers include ceteareth-10 to -25, ceteth-10-25, steareth-10-25 (i.e. C16 to C18 alcohols ethoxylated with 10 to 25 ethylene oxide residues) and PEG-15-25 stearate or distearate. Other suitable examples include C10-C20 fatty acid mono, di or tri-glycerides. Further examples include C18-C22 fatty alcohol ethers of polyethylene oxides (8 to 12 EO).

Examples of emulsifiers, which typically have a low HLB value, often a value from 2 to 6 are fatty acid mono or possibly diesters of polyhydric alcohols such as glycerol, sorbitol, erythritol or trimethylolpropane. The fatty acyl moiety is often from C14 to C22 and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid.

A particularly desirable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers also include C1 to C12 alkyl groups as functional groups. Examples of suitable surfactants include DC5225 and DC 5200 (from Dow Corning), Abil EM 90 and EM 97 (from Gold Schmidt) and KF 6026, KF 6028, KF 6038 (from Shinetsu Silicones).

The skilled artisan should appreciate that other emulsifying surfactants than those described above may also be used in antiperspirant compositions described herein.

D. Formation of the Emulsion

The continuous phase, disperse phase, and emulsifying surfactant are combined and then mixed or otherwise agitated sufficiently to form an emulsion. Typically, the disperse phase is added slowing to the continuous phase while the continuous phase is being vigorously agitated with a mixing system. The skilled artisan should appreciate the degree of mixing needed based on the desired phase ratio of the emulsion, its resulting viscosity and the desired batch size. The resulting emulsion can be further processed to create a consistent droplet size within the emulsion; for example, the emulsion may be processed by a mill to reduce droplet size and/or improve droplet size uniformity. Preferably, the emulsion is processed so that the entire batch experiences an equivalent amount of shear. A single-phase inline mill is one preferred apparatus for the additional, optional processing.

E. Optional Ingredients

Antiperspirant compositions of the present invention may include one or more fragrance/perfume materials. In one preferred embodiment, the composition includes a fragrance material comprising a plurality of different perfume raw materials. Typical perfume levels in the present invention are 0.25 to 5%. Nonlimiting examples of fragrance materials include any known fragrances in the art or any otherwise effective fragrance materials. Typical fragrances are described in Arctander, “Perfume and Flavour Chemicals (Aroma Chemicals)”, Vol. I and II (1969) and Arctander, “Perfume and Flavour Materials of Natural Origin” (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314), aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the antiperspirant compositions may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof. Fragrance materials other than those listed above may also be employed.

The antiperspirant compositions can also include residue-masking agents to reduce the appearance of white residue arising from the antiperspirant active and structurant employed in the product. These masking agents can be incorporated into either the continuous or disperse phased depending on their water solubility. Exemplary residue-masking agents include isostearyl isostearate, glycereth-7-benzoate, C12-C15 alkyl benzoate, octyldodecyl benzoate, isostearyl lactate, isostearyl palmitate, benzyl laurate, laureth 4, laureth 7, oleth 2, PEG 4, PEG 12, isopropyl myristate isopropyl palmate, butyl stearate, polyethylene glycol methyl ethers, PPG 2 ceteareth 9, PPG 2 isodeceth 12, PPG 5 butyl ether, PPG 14 butyl ether, PPG 15 butyl ether, PPG 53 butyl ether, octyldodecanol, polydecene, mineral oil, petrolatum, phenyltrimethicone, dimethicone copolyol, and mixtures thereof. One preferred concentration level of the optional residue-masking agent is from about 3% to about 10%, by weight of the composition. But other concentration levels may also be used.

Antiperspirant compositions of the present invention may employ one or more additional ingredients. Nonlimiting examples of such optional ingredients include, but are not limited to, pH buffering agents, additional malodor controlling agents, emollients, humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), and lichen extract. Many of these optional ingredient categories can include natural or plant-derived materials. For example, emollients can be selected from the group comprising almond oil, avocado oil, coconut oil, hazelnut oil, jojoba oil, olive oil, palm oil, pumpkin seed oil, safflower oil, sesame oil, sunflower oil, tamanu oil, and wheat germ oil. And colorants can be derived from materials such as, for example, annatto, beets, blueberries, caramel, carrot powder, elderberries, grape juice, paprika, raspberries, red cabbage, saffron, spinach powder, tomato powder, and turmeric.

### III. Underarm Product Line-Ups

The present invention is also directed to underarm product line-ups. The product line-ups include at least one antiperspirant product and at least one deodorant product. One exemplary underarm product line-up comprises (a) an antiperspirant product comprising a dispenser that includes a body and a cap, the body including a transparent section; and an antiperspirant composition disposed in the dispenser that is visible through the transparent section, the antiperspirant composition comprising an antiperspirant active and being translucent to opaque in appearance; and (b) a deodorant product comprising a dispenser that includes a body and a cap, the body including a transparent section; and a deodorant composition disposed in the dispenser that is visible through the transparent section, the deodorant composition being transparent to translucent in appearance; wherein the antiperspirant product and the deodorant product are from the same manufacturer.

Another underarm product line-up comprises (a) an antiperspirant product comprising a first dispenser that includes a first body and a first cap; and an antiperspirant composition disposed in the dispenser, the antiperspirant composition comprising an antiperspirant active and being translucent to opaque in appearance; and (b) a deodorant product comprising a second dispenser that includes a second body and a second cap, the second body including a transparent section; and a deodorant composition disposed in the dispenser that is visible through the transparent section, the deodorant composition being transparent to translucent in appearance; wherein the first body and the second body are the same color, and wherein the antiperspirant product and the deodorant product are from the same manufacturer.

Exemplary antiperspirant products and compositional aspects of the same have been described above. Exemplary deodorant compositional aspects are described in greater detail below and can be formulated as an aqueous (including from about 10% to about 60% water) or anhydrous composition (including from less than about 10%, less than about 3%, less than about 1%, or zero percent water).

Deodorant compositions of the present invention comprise an emollient system including at least one, and preferably a combination of different emollients. The emollients are preferably liquid under ambient conditions, and comprise at least one emollient having a solubility parameter greater than about 9 and more preferably greater than about 11, and a vapor pressure below about 2 mm Hg at 25° C. Depending on the type of product form desired, concentrations of the emollient(s) in the deodorant compositions can range from about 10% to about 90%, and preferably from about 25% to about 75%, by weight of the deodorant composition.

Emollients suitable for use in the deodorant compositions of the present invention include, but are not limited to, topically safe and effective organic, silicone-containing or fluorine-containing, volatile or non-volatile, polar or non-polar, provided that the resulting combinations of emollients form a solution or other homogenous liquid or liquid dispersion at the selected processing temperature of the composition. Processing temperatures for the deodorant compositions can range from about 15° C. to about 150° C., depending on product form. Process temperatures for stick deodorant products typically range from about 50° C. to about 100° C. Preferred emollients with the requisite solubility parameter and volatility include but are not limited to: propylene glycol, dipropylene glycol, tripropylene glycol, diethylene glycol, triethylene glycol, PEG-4, PEG-8, 1,2 pentanediol, 1,2 hexanediol, hexylene glycol, and glycerin. Applicant has discovered that, in some product forms, blends of these emollients are most preferred.

Other emollients can be included in the product to provide other benefits to the deodorant such as, for example, good skin feel or skin moisturization. Other examples of suitable emollients include C2 to C20 monohydric alcohols, C2 to C40 dihydric or polyhydric alcohols, alkyl ethers of polyhydric and monohydric alcohols, volatile silicone emollients such as cyclopentasiloxane, non volatile silicone emollients such as dimethicone, mineral oils, polydecenes, and petrolatum.

Deodorant compositions of the present invention may comprise a suitable thickening or structuring agent in order to provide the desired hardness and application characteristics to the compositions. The thickening or structuring agent concentrations may range from about 0.1%, to about 30%, by weight of the composition depending on the type of product and thickening or structuring agent. Preferred inclusion ranges for stick products are from about 4 to about 20%.

Aqueous deodorant compositions of the present invention may comprise a thickening or structuring agent that can melt to form a solution or other homogenous liquid or liquid dispersion within the liquid carrier at a processing temperature of from about 50° C. or to about 150° C., preferably from about 60° C. to about 100° C.

Suitable thickening or structuring agents for use in the aqueous and anhydrous deodorant compositions of the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, triglycerides, and other suitable gellants. Other examples include finely divided or colloidal silicas, fumed silicas, and silicates, which includes montmorillonite clays and hydrophobically treated montmorillonites, e.g., bentonites, hectorites and colloidal magnesium silicates.

Preferred thickening or structuring agents for use in the aqueous and anhydrous deodorant compositions are the solid salts of fatty acids wherein the fatty acid moiety has from about 12, from about 16 or from about 18 carbon atoms to about 40, to about 22, or about 20 carbon atoms. Suitable salt forming cations for use with these thickening or structuring agents include metal salts such as alkali metals (e.g. sodium and potassium), alkaline earth metals (e.g. magnesium), and aluminum. Preferred are sodium and potassium salts. For example, suitable salt forming cations may be selected from the group consisting of sodium stearate, sodium palmitate, potassium stearate, potassium palmitate, sodium myristate, aluminum monostearate, and combinations thereof.

The deodorant compositions typically employ fragrance materials for imparting a pleasant odor and/or masking malodor. The deodorant compositions may also include deodorant actives, which encompasses any topical material that is known or otherwise effective in preventing or eliminating malodor associated with perspiration. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

The deodorant compositions of the present invention may further comprise one or more optional components which may modify the physical or chemical characteristics of the compositions or serve as additional “active” components when deposited on the skin. Of course, such optional components may be included provided that they are physically and chemically compatible and do not otherwise unduly impair product stability, aesthetics, or performance. Nonlimiting examples of such optional materials include, but are not limited to, pH buffering agents, additional malodor controlling agents such as deodorant actives, fragrance materials, emollients, humectants, soothing agents, dyes and pigments, medicaments, baking soda and related materials, preservatives, and soothing agents such as aloe vera, allantoin, D-panthenol, avocado oil and other vegetative oils, and lichen extract.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method of merchandising an antiperspirant product, comprising the steps of:
a) offering a solid or cream antiperspirant product for sale, wherein the antiperspirant product is a water-in-oil emulsion comprising 5% or more of water and one or more plant-derived materials in the continuous oil phase; and
b) communicating to perspective buyers that the antiperspirant product is natural and/or provides an environmental benefit.

2) The method of claim 1, wherein the one or more plant-derived materials is a colorant.

3) The method of claim 1, wherein the one or more plant-derived materials is a rheology-modifying material.

4) The method of claim 1, wherein the one or more plant-derived materials makes up more than 2%, by weight, of the antiperspirant product.

5) The method of claim 1, wherein the communication in step (b) comprises a term(s) selected from the group consisting of natural, nature, plant, vegetable, environment, eco, green, pure, purity, not artificial, organic, not man-made, petroleum free, being, wellness, and health.

6) A method of merchandising an antiperspirant product, comprising the steps of:
a) offering an antiperspirant product for sale, the antiperspirant product comprising an antiperspirant active containing a metal salt and less than about 30% volatile silicone that is not labeled CEPA toxic; and
b) communicating to perspective buyers, in conjunction with step (a), that the antiperspirant product is natural and/or provides an environmental benefit.

7) The method of claim 6, wherein the antiperspirant product comprises from about 1% to about 4% aluminum.

8) The method of claim 6, wherein the communication in step (b) comprises a term(s) selected from the group consisting of natural, nature, plant, vegetable, environment, eco, green, pure, purity, not artificial, organic, not man-made, petroleum free, being, wellness, and health.

9) The method of claim 6, wherein the antiperspirant product is a water-in-oil emulsion.

10) The method of claim 9, wherein the antiperspirant product is in the form of a solid stick.

11) The method of claim 6, wherein the antiperspirant product is a liquid.

12) The method of claim 6, wherein the antiperspirant product is anhydrous.

13) An antiperspirant, comprising:
a) a continuous oil phase;
b) a discontinuous water phase comprising an antiperspirant active including a metal salt; and
c) from about 0.0001% to about 2%, by weight, of a plant-derived colorant;
wherein the antiperspirant is in the form of a cream or solid stick, and wherein the antiperspirant is not white or clear.

14) The antiperspirant of claim 13, wherein the continuous oil phase comprises less than about 30% volatile silicone that is not labeled CEPA toxic.

15) The antiperspirant of claim 13, wherein the continuous phase comprises one or more plant-derived oils.

16) The antiperspirant of claim 13, wherein the discontinuous water phase comprises from about 1% to about 4% aluminum.

17) The antiperspirant of claim 13, further comprising a plant-derived rheology modifying agent.

18) The antiperspirant of claim 13, further comprising packaging that includes indicia communicating to perspective buyers that the antiperspirant is natural and/or provides an environmental benefit.

19) The antiperspirant of claim 18, wherein the indicia comprises a non-text symbol or graphic connoting organic, plant, vegetable, green, eco, or nature.

20) The antiperspirant of claim 18, wherein the indicia comprises a term(s) selected from the group consisting of natural, nature, plant, vegetable, environment, eco, green, pure, not artificial, organic, not man-made, petroleum, being, wellness, and health.
